(19) United States
(12) Patent Application Publication
(10) Pub. No.: US 2009/0209497 A1
(43) Pub. Date: Aug. 20, 2009

(54) METHOD OF TREATING LYMPHANGIOLEIOMYOMATOSIS (LAM)

(76) Inventors: Judah Folkman, Brookline, MA (US); George Abrams, Newton, MA (US); Marsha A. Moses, Brookline, MA (US)

Correspondence Address: WOLF GREENFIELD & SACKS, P.C., 600 ATLANTIC AVENUE, BOSTON, MA, 02210-2206, US

(21) Appl. No.: 11/913,709

(22) PCT Filed: May 16, 2006
(86) PCT No.: PCT/US06/19020

§ 371 Date: Nov. 6, 2008

Related U.S. Application Data
(60) Provisional application No. 60/681,412, filed on May 16, 2005.
(60) Provisional application No. 60/778,306, filed on Mar. 1, 2006.

(51) Int. Cl. A61K 31/65; A61P 11/00

(52) U.S. Cl. 514/154

(57) ABSTRACT

The invention provides a method for the treatment of Lymphangioleiomyomatosis (LAM) in the human subject in need thereof. The method comprises administering to the subject an effective amount of doxycycline or a salt thereof. The invention further provides a method for monitoring the efficacy of treatment. Treatment efficacy is monitored by measuring MMP levels. Reduction in MMP levels indicates that the treatment is effective.

### CROSS-REFERENCE TO RELATED APPLICATIONS

The present application claims priority to U.S. provisional application Ser. No. 60/681,412 filed on May 16, 2005 and U.S. provisional application Ser. No. 60/778,306 filed on Mar. 1, 2006, the contents of which are incorporated herein by reference.

### BACKGROUND OF THE INVENTION

Lymphangioleiomyomatosis (LAM) is a progressive lung disease. The disease is characterized by proliferation of normal smooth muscle cells, usually designated as LAM cells, in pulmonary interstitium along the axial lymphatics of thorax and abdomen. Over time, the smooth muscle cells block the flow of air, blood and the lymph to and from the lungs preventing the lungs from functioning properly.

There is currently no successful treatment for LAM.

### SUMMARY OF THE INVENTION

The present invention provides a method for the treatment of lymphangioleiomyomatosis (LAM). The method comprises administering to a subject in need thereof an effective amount of doxycycline or a salt thereof.

The invention further provides a method for monitoring the efficacy of treatment for LAM by the methods of the present invention. The method comprises measuring the level of at least one MMP after a first period of time after treatment. The first period of time may be greater than 10 days, greater than 20 days or greater than 30 days. The MMP may be selected from the group consisting of an MMP greater than 150 kDa, 125 kDa, 92 kDa and 72 kDa. The MMP may be complexed with NGAL. At least two MMPs may be measured. At least three MMPs may be measured.

The method may further comprise measuring the level of at least one MMP before treatment, wherein a decrease in the MMP level after treatment indicates that the treatment is effective.

The present invention also provides for the use of doxycycline or a salt thereof in the preparation of a medicament for the treatment of LAM.

### BRIEF DESCRIPTION OF THE DRAWINGS

FIGS. 1A-1B show successful treatment of LAM with doxycycline: monitoring therapeutic efficacy and measuring clinical improvement. FIG. 1A shows quantitative analysis of MMPs in patient's urine demonstrating significant reduction in urinary MMP activity during course of doxycycline treatment. FIG. 1B shows prolonged O₂ saturation during course of therapy; increased time to desaturation to 90% O₂ after removal of supplemental O₂ (left panel) and sustained O₂ saturation after exercise while on doxycycline therapy (right panel; representative results from clinically-administered 6-min walk test). Data points represent single measurements taken prior to or during doxycycline therapy.

### DETAILED DESCRIPTION OF THE INVENTION

The present inventors have surprisingly discovered that LAM can be treated by administration of doxycycline. Accordingly, the invention provides a method for treating LAM comprising administering to a subject in need thereof an effective amount of doxycycline or a salt thereof.

For use in the method of present invention doxycycline is preferably formulated for oral or inhalation administration. The dose of administration may be lower than is necessary for use as an antibiotic. However, higher doses may also be used. The amount of doxycycline to be administered will be determined by the usual factors such as the nature and severity of the disease and the condition of the patient.

In one embodiment, it is advantageous to formulate doxycycline in devices suitable for pulmonary delivery and deliver them topically to the lung. This can be achieved using a range of pulmonary systems and formulation techniques known to those skilled in the art such as, but not limited to, nebulizers, multiple-dose inhalers, dry powder inhalers, and pressurized metered multi-dose inhalers.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

It is especially advantageous to formulate the doxycycline compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Doxycycline is preferably orally administered at a dose of 20 milligrams to 400 milligrams per day. In one embodiment, the dose is below 200 milligrams per day. In an alternative embodiment, the dose of doxycycline is 40 milligrams or less per day. In yet another embodiment, the dose of doxycycline is 100 milligrams or less per day.

The invention further provides a method of diagnosing LAM, confirming the diagnosis of LAM, and monitoring the efficacy of the treatment. Each of these methods involve measuring MMP levels in a biological sample from the patient, e.g., a urine sample, using, for example, the methods set forth in Moses et al., U.S. Pat. No. 6,811,995 and WO 02/31507, the disclosures of which are incorporated herein by reference. In the method of monitoring treatment, one would look for changes in levels of at least one of the following MMPs: greater than 150 kDa, approximately 92 kDa (MMP-9), 72 kDa (MMP-2) and an MMP complexed with a lipocalin, e.g., approximately 125 kDa (MMP-9/NGAL complex), after administration of the doxycycline. A decrease in the level of at least one MMP indicates that the treatment is effective. Preferably, one would look at least 2, 3 or more MMPs, including MMPs complexed with lipocalins, e.g., NGAL. Preferably, one would look at MMP-2, MMP-9 and/or a MMP-9/NGAL complex. If the MMP level(s) does not decrease after administration, e.g., after one month, the dose of doxycycline is increased and the MMP level(s) measured again. This can continue until the proper dose is determined. The MMP monitoring can continue throughout to ensure that the treatment is still effective. For example, the patient can be monitored monthly.

In the method of diagnosis, one would look for the presence of MMPs in a biological sample (e.g., urine) of a patient. In one embodiment, the patient is one suspected of having LAM and the presence of MMPs in the biological sample is confirmatory. Symptoms of LAM include: dyspnea—shortness of breath; hemoptysis—coughing up blood-stained sputum or blood; chylous effusions—leakage of white fluid into the chest cavity; and repeated pneumothoraces—leakage of air into the chest cavity.

The levels of MMPs can be measured by any means known to those skilled in the art, for example methods disclosed in U.S. Pat. No. 6,811,995 and U.S. Pat. Appl. Nos. 20020081641, 20030215900, the contents of which are herein incorporated by reference. In the present invention, it is generally preferred to use antibodies, or antibody equivalents, to detect MMP levels. However, other methods for detection can also be used. For example, MMP levels may be monitored by mass spectrometric analysis.

The term “biological sample” includes biological samples obtained from a subject. Examples of such samples include urine, blood taken from a prick of the finger or other source such as intravenous, blood fractions such as serum and plasma, feces and fecal material and extracts, saliva, cerebrospinal fluid, amniotic fluid, mucus, and cell and tissue material such as cheek smear, Pap smear, fine needle aspiration, sternum puncture, and any other biopsied material taken during standard medical and open surgical procedures. In one preferred embodiment, the biological sample is a urine sample.

In using an electrophoretic technique for separation of enzymes, e.g., SDS-PAGE, the electrophoretogram may be developed as a zymogram. The term “zymography” is meant here to include any separations system utilizing a chemically inert separating or support matrix that allows detection of an enzyme following electrophoresis, by exposing the matrix of the separations system to conditions that allow enzyme activity and subsequent detection. More narrowly, the term zymography designates incorporation of an appropriate substrate for the enzyme of interest into the inert matrix, such that exposing the matrix to the conditions of activity after the electrophoresis stop yields a system to visualize the precise location, and hence the mobility, of the active enzyme. By techniques well-known to the skilled artisan, the molecular weights of proteins are calculated based on mobilities derived from positions on a zymogram. Such techniques include comparison with molecular weight standards, the mobilities of which are determined from general protein stains or from pre-stains specific to those standards, and comparison with positive controls of purified isolated enzymes of interest, which are visualized by the technique of the zymogram, i.e., enzyme activity.

In particular, substrates for detection of proteases by zymography are included in the electrophoresis matrix. For MMPs, i.e., type IV collagenases, the natural substrate is a type IV collagen and gelatin, a type I collagen derivative, used for the zymography substrate. However other proteins that are suitable for detection of further proteases of interest in LAM diagnosis, for example, include fibronectin; vitronectin; collagens of types I through III and V through XII; procollagens; elastin; laminin; plasmin; plasminogen; entactin; nidogen; syndecan; tenascin; and sulfated proteoglycans substituted with such saccharides as hyaluronic acid, chondroitin-6-sulfate, condroitin-4-sulfate, heparan sulfate, keratan sulfate, and dermatan sulfate and heparin. Further, convenient inexpensive substrate proteins such as casein, which may not be the natural target of a protease of interest, but are technically appropriate, are included as suitable substrate components of the zymography techniques of the present invention. Chemically synthesized mimetics of naturally occurring protein substrates are also potential zymography substrates, and may even be designed to have favorable properties, such chromogenic or fluorogenic ability to produce a color or fluorescent change upon enzymatic cleavage.

The zymogram may be developed by use of a general stain for protein, e.g., Coomassie Blue dye, Amido Black dye, and SYPRO Orange stain (Biorad Laboratories, Hercules, Calif.). Further, enzyme activity may be detected by additional techniques beyond that of a clear zone of digestion in a stained matrix, for example, by absence of areas of radioactivity with a radio-labeled substrate, by change in mobility of a radio-labeled substrate, or by absence of or change in mobility of bands of fluorescence or color development with use of fluorogenic or chromogenic substrates, respectfully.

Quantitative densitometry can be performed with zymograms by placing the gel directly on an activated plate of a Molecular Dynamics phosphorimager (Molecular Dynamics, Sunnyvale, Calif., or with a Datacopy G8 plate scanner attached to a MacIntosh computer equipped with an 8-bit videocard and McImage (Xerox Imaging Systems). Background measurements, areas of the gel separate from sample lanes, can similarly be scanned, and values subtracted from the readings for enzyme activities.

Another electrophoretically-based technique for analysis of a biological sample for presence of specific proteins is an affinity-based mobility alteration system (Lander, A. (1991) Proc Natl Acad Sci USA, 88(7):2768-2772). An MMP or MMP-complex might be detected, for example, by inclusion of a substrate analog that binds essentially irreversibly to the enzyme, hence decreasing the mobility. The affinity material is present during electrophoresis, and is incorporated into the matrix, so that detection of the enzyme of interest occurs as a result of alteration of mobility in contrast to mobility in the absence of the material. Yet another technique of electrophoretic protein separation is based on the innate charge of a protein as a function of the pH of the buffer, so that for any protein species, there exists a pH at which that protein will not migrate in an electric field, or the isoelectric point, designated pI. Proteins of a biological sample, such as a urine sample, may be separated by isoelectric focusing, then developed by assaying for enzymatic activity for example by transfer to material with substrate, i.e., zymography. Electrophoresis is often used as the basis of immunological detections in which the separation step is followed by physical or electrophoretic transfer of proteins to an inert support such as paper or nylon (known as a “blot”), and the blotted pattern of proteins may be detected by use of a specific primary binding (Western blot) by an antibody followed by development of bound antibodies by secondary antibodies bound to a detecting enzyme such as horse radish peroxidase. Additional immunological detection systems for LAM associated MMP enzyme complexes are now described in detail below.

In one embodiment, levels of MMP proteins are measured by contacting the biological sample, e.g., urine sample, with an antibody-based binding moiety that specifically binds to the MMP, or to a fragment of MMP. Formation of the antibody-MMP complex is then detected as a measure of MMP levels.

In the methods of the invention that use antibody based binding moieties for the detection of MMP, the levels of MMP proteins present in the biological samples, e.g., urine samples, correlate to the intensity of the signal emitted from the detectably labeled antibody.

The antibody-based binding moiety is detectably labeled by linking the antibody to an enzyme, e.g., horseradish peroxidase, alkaline phosphatase. The enzyme, in turn, when exposed to its substrate, will react with the substrate in such a manner as to produce a chemical moiety which can be detected, e.g., by spectrophotometric, fluorometric, chemiluminescent or by visual means. Alternatively, the antibody may be labeled radioactively, e.g., 3H, 131I, 35S, 14C, 125I; with a fluorescent compound e.g., fluorescein isothiocyanate, rhodamine, phycoerytherin; with a fluorescent emitting metal e.g., 152Eu, or others of the lanthanide series, or by coupling with a chemiluminescent compound, e.g., luminol, luciferin, isoluminol. Methods for the detection of the presence of these labeling agents are known to the skilled artisan.

“Radioimmunoassay” is a technique for detecting and measuring the concentration of an antigen, e.g., MMPs, using a labeled (e.g., radioactively labeled) form of the antigen. Examples of radioactive labels for antigens include 3H, 14C, and 125I. The concentration of antigen MMP in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, e.g., sandwich ELISA or competitive ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies, e.g., luciferase, alkaline phosphatase, horseradish peroxidase, or beta-galactosidase, or chemical methods, e.g., DAB/Substrate chromagen.

Other techniques may be used to detect MMPs, according to a practitioner's preference, e.g., western blotting (Towbin et al., Proc. Nat. Acad. Sci. 76:4350 (1979)). Antibody arrays or protein chips can also be employed, see for example U.S. Patent Application Nos.: 20030013208A1; 20020155493A1; 20030017515 and U.S. Pat. Nos. 6,329,209; 6,365,418, which are herein incorporated by reference in their entirety.

In addition, MMPs may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos.: 20030199001, 20030134304, 20030077616, which are herein incorporated by reference.

The antibodies for use in the present invention can be obtained from a commercial source, e.g., Chemicon Int'l Inc., Temecula, Calif.; QED Bioscience Inc., San Diego, Calif. Alternatively, antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

All references cited above or below are herein incorporated by reference.

The present invention is further illustrated by the following Example. The Example is provided to aid in the understanding of the invention and is not construed as a limitation thereof.

### EXAMPLE

Lymphangioleiomyomatosis (LAM), a rare lung disease typically affecting women of reproductive age, is characterized by abnormal proliferation of smooth muscle cells and progressive loss of pulmonary function due to tissue destruction (1). Long-term survival is rare; LAM patients often succumb to respiratory failure within 10 years of diagnosis. Currently no standard-of-care therapy exists for LAM, although limited success has been reported with hormone therapy or lung transplantation.

Destruction of lung parenchyma is attributed to a pro-proteolytic environment resulting from increased expression and activity of matrix metalloproteinases (MMPs). MMP-2, in particular, is significantly up-regulated in LAM pulmonary tissues (2). We have treated pulmonary capillary hemangiomatosis (3) with the antibiotic doxycycline, an MMP inhibitor (4). We hypothesized that doxycycline treatment of LAM would inhibit MMP-induced tissue degradation, thereby providing clinical benefit. Having previously reported that the presence of urinary MMPs is a predictor of disease status in patients with diseases characterized by dysregulated ECM degradation such as cancer (5) and hemangiomatosis (3), we monitored the therapeutic efficacy of doxycycline treatment for LAM by analyzing urinary MMP profiles during the course of treatment. Here, we present a patient with LAM who experienced substantial and rapid clinical improvement after doxycycline therapy.

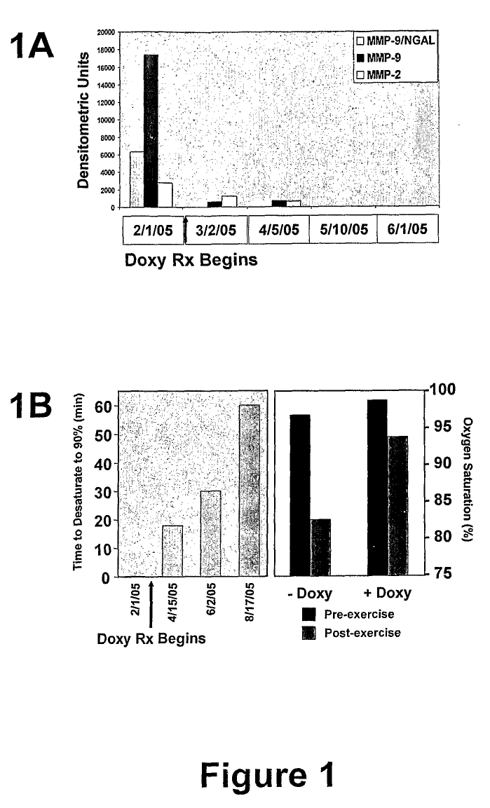

A 66 year-old woman was diagnosed with LAM in 1975 after a partial nephrectomy for angiomyolipoma. Although stable for many years, her condition had deteriorated with an FEV1 as low as 0.48 liters (21 percent of predicted), and she was placed on the lung transplant list. Prior to doxycycline treatment, several MMP species were significantly elevated in the patient's urine, including MMP-2, MMP-9 and a MMP-9/NGAL complex (FIG. 1A). Based on these elevated MMP levels, doxycycline was initiated at an initial dose of 20 mg/day. Dose escalation (up to 100 mg/day) was determined by monthly urinary MMP profiling (FIG. 1A) and how well the drug was tolerated. Lung capacity increased (recent FEV1 is 0.91 liters, 35 percent of predicted) and enhanced O₂ saturation was observed (FIG. 1B). Telegraphic speech, which was present, resolved. The patient has had clear improvement in quality of life and was removed from the lung transplant list, as predicted by the decreasing levels of urinary MMPs (FIG. 1A).

This case supports the hypothesis that doxycycline, guided by urinary MMP monitoring, may represent a promising therapy for the treatment of LAM, and represents a potential means to ameliorate this disease by treating the biomarker, in this case, urinary MMP levels.

All references cited throughout the application are incorporated herein by reference.

### REFERENCES

1. Sullivan E J. Lymphangioleiomyomatosis: a review. Chest 1998; 114: 1689-703.
2. Matsui K. Takeda K, Yu Z-X, Travis W D, Moss J. Ferrans V J. Role for activation of matrix metalloproteinases in the pathogenesis of pulmonary lymphangioleiomyomatosis. Arch Pathol Lab Med 2000; 124: 267-75.
3. Ginns L C, Roberts D H, Mark E J, Brusch J L, and Marler J J. Pulmonary capillary hemangiomatosis with atypical endotheliomatosis: successful antiangiogenic therapy with doxycycline. Chest 2003; 123: 2017-22.
4. Schmeider B S, Maimon J. Golub L M, Ramamurthy N S, Greenwald R A. Tetracyclines inhibit intracellular muscle proteolysis in vitro. Biochem Biophys Res Comm 1992; 188: 767-72.
5. Moses M A, Wiederschain D, Loughlin K R, Zurakowski D, Lamb C C and Freeman M R. Increased incidence of matrix metalloproteinases in urine of cancer patients. Cancer Res 1998; 58: 1395-9.

1. A method for the treatment of Lymphangioleiomyomatosis (LAM) in the human subject in need thereof, comprising administering to the subject an effective amount of doxycycline or a salt thereof.

2. The method of claim 1, wherein the amount of doxycyline administered is between 20 milligrams to 400 milligrams per day.

3. The method of claim 1, wherein the amount of doxycycline is 40 milligrams or less per day.

4. The method of claim 1, wherein the amount of doxycycline is 100 milligrams or less per day.

5. The method of claim 1, further comprising measuring the level of at least one matrix metalloproteinase (MMP) after a first period after treatment.

6. The method of claim 5, further comprising measuring the level of at least one MMP before treatment, wherein a decrease in the MMP level after treatment indicates that the treatment is effective.

7. The method of claim 5, wherein the first time is greater than 10 days, greater than 20 days or greater than 30 days.

8. The method of claim 5, wherein the MMP is selected from the group consisting of an MMP greater than 150 kDa, an MMP of approximately 125 kDa, an MMP of approximately 92 kDa, and an MMP of approximately 72 kDa.

9. The method of claim 5, wherein the MMP is complexed with NGAL.

10. The method of claim 7, wherein at least 2 MMPs are measured.

11. (canceled)

12. A method for confirming a diagnosis of Lymphangioleiomyomatosis (LAM) in a patient suspected of having LAM, comprising obtaining a urine sample from the patient; and detecting the presence of a matrix metalloproteinase (MMP) in the urine sample, wherein the presence of the MMP is indicative of the presence of LAM.

13. (canceled)

14. The method of claim 12 wherein the MMP is selected from the group consisting of an MMP greater than 150 kDa, an MMP of approximately 125 kDa, an MMP of approximately 92 kDa, and an MMP of approximately 72 kDa.

15. The method of claim 12 wherein the MMP is complexed with NGAL.

16. A method for assessment of lymphangioleimyomatsis (LAM), the method comprising:
(a) assaying for the presence of at least one matrix metalloproteinase (MMP) in a sample obtained from a subject; and
(b) determining whether the at least one MMP is present at a level higher than a predetermined level,
thereby indicating whether the subject has or is at risk of developing LAM.

17. The method of claim 16 wherein the sample is a urine sample.

18. The method of claim 16 wherein the predetermined level is based on the level of at least one MMP normally found in biological samples of healthy subjects.

19. The method of claim 16 wherein the predetermined level is based on at least one of the subject's MMP levels prior to treatment.

20. The method of claim 16 wherein at least one of the subject's MMP levels is monitored over time.

21. The method of claim 16 wherein the at least one MMP is selected from the group consisting of an MMP greater than 150 kDa, an MMP of approximately 125 kDa, an MMP of approximately 92 kDa, and an MMP of approximately 72 kDa.

22. The method of claim 16 wherein the at least one MMP is complexed with NGAL.

23. A method for assessment of lymphangioleimyomatsis (LAM) treatment, the method comprising:
(a) assaying for the presence of at least one matrix metalloproteinase (MMP) in a sample obtained from a subject having LAM before treatment of LAM; and
(b) determining whether the at least one MMP is present at a level lower after treatment,
thereby indicating the treatment is effective.

24. The method of claim 23 wherein the treatment comprises administering to the subject an effective amount of doxycycline or a salt thereof.

25. The method of claim 23 wherein the sample is a urine sample.

26. The method of claim 23 wherein the at least one MMP is selected from the group consisting of an MMP greater than 150 kDa, an MMP of approximately 125 kDa, an MMP of approximately 92 kDa, and an MMP of approximately 72 kDa.

27. The method of claim 23 wherein the at least one MMP is complexed with NGAL.
